(19) United States
(12) Patent Application Publication
(10) Pub. No.: US 2005/0142230 A1
(43) Pub. Date: Jun. 30, 2005

(54) Pharmic activity and manufacturing method of the production produced by six kinds of plant compound

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/746,372

(22) Filed: Dec. 29, 2003

(51) Int. Cl. A61K 35/78

(52) U.S. Cl. 424/746; 424/774; 424/777

(57) ABSTRACT

The certified doctor in United States, use pentobabital sodium as the hypnotic to refrain central nervous system. Pentobabital sodium can be patience to the body after two weeks, and the effect decrease half, and have lingering effects and remain effects. The making method and pharmacological activity about the six kinds of plants and sodium pentobarbital's synergistic action wares are gaven patent. Under the cautious and strict conditions, extract the plant ingredients, absorb them by WLD resin, separate and purify them, and use the finger mark atlas quantity to make sure that the end product poise and stabilize. Six plant compound wares have sedate, hypnotize and synergy pentobabital sodium, anti-penfourzopol. The clinical use proved the reliability. It is safe and compensatory to the pentobabital sodium. It solves the hypnotic's tolerance and addiction. It solves the problem, which is expected to conquer for a long time.

The pharmic activity and manufacturing method of the production produced by six kinds of plant compound (including Radix Salviae Miltiorrhizae, Radix Rehmanniae, Rhizoma Atractylodis Macrocephalae, Radix Polygoni Multiflori, Radix Polygalae, Fructus Jujubae) and pentobabital together. (in brief the pharmic activity and manufacturing method of the production produced by six kinds of plant compound and pentobabital together)

### BACKGROUND OF THE INVENTION

The pharmic activity and manufacturing method of the production cooperated by six kinds of plant compound and pentobabital together is a kind of combination patent of American patents which is combined by the methods of natural production pharmic activity and separation, purification natural production. The study of pharmic activity of the six kinds of plant compound confirms that the compound production are of better sedation, and have cooperated effect with hypnosis mete under pentobabital threshold with distinct hypnosis effect and effect of anti pentylene tetrazol convulsion.

The America provides patent protection not only for medication used in disease treatments, but also for methods of disease treatments. The conclusion of pharmic activity experiment cooperated by American patents and brands satisfies the relevant requirements of American Exclusive Law.

The sedation hypnosis medicine is a kind of medicine restraining nerve center system. The sedation medicine decreases excitability of nerve center system, and makes the taker in a state of quiet and sleep. It is mostly used in treating anxiety, fret and neurosis. The hypnosis medicine could cause narcolepsy, produce and maintain a state similar to physical sleep, it is used to treat insomnia. However, there is no distinct difference between them. Generally speaking, small dose of hypnosis medicine has the effect of sedation, medium of which could cause hypnosis, and the dose more than that of hypnosis has the effect of anti convulsion and narcosis.

The periodic change of disillusion and sleep and the checking directory of superior departments and corresponding court prejudication have all explained for the requirements of pharmic practicability. If an invention claims to have the practicability to treat man or animal, then it must be judged whether the practicability meets the practicability requirements specified by Exclusive Law, and whether the compatible standard is the same one with inventions in other technical field. And it is particularly emphasized that the practicability requirements of treatment in Exclusive Law shouldn't be confused with the requirements of FDA about safety and effect of the relevant medicine that came in the American market. The basement of judging the practicability of such kind of invention is to invent the activity of the remedial, prophylactic and pharmic medicine. The effect cooperated by six kinds of plant compound and pentobabital together is necessary for the normal physical function of animal nerve center system. The insomnia is reletively shortage of sleep quantity and quantity which occurs commonly. There is about one third American people have certain kind of sleep difficulty. Difficult to sleep and to awake earlier, including which the chronic insomnia accounts for 20%. Sleep difficulty is general the result of diversified complex factors, psychology, environment, caducity and taking and not taking of some medicine produce side effect to sleep. As older and more insomnia the elders sleep less and rarely have deep sleep of thee or four period. The increase of common sick and decrease of physical function and the dread of coming death are important reason for elders insomnia. When the insomnia turns chronically, the psychopathology is an important reason. The insomnia and dread to it easily establishes a malign circle enabled in physiology, insomnia, fear of insomnia, further insomnia. The most effective way to solve this question is to use hypnosis medicine.

Pentobabital is a kind of hypnosis medicine acted on nerve center system. Pentobabital restrains all the excited organizations, especially sensitive to nerve center system. Hypnosis dose of pentobabital could increase total sleep time and change sleep period including shorten sleep latent period, and decrease awaking times, shorten FWS and SWS time. Taking the medicine repeatedly could produce medicine-proof in several days, if taking the medicine for two weeks, the effect to total sleep time will decrease a half, if stop to taking the medicine will rebound and increase FWS distinctly.

If taking the pentobabital at night, someone will swirl, dose and feel hyponoia, weaken the ability of judgement and operation, sometimes feel excited excessively, energetic, easily angry, these symptom of pentobabital are called residual effects. Some patients feel excited as drunken but not being restrained, these special reactions are common in elders and sicks.

The clinical observation of the production produced by six kinds of plant compound and pentobabital together indicates that during treatment we haven't found patients in observation groups who took the compound production appeared reactions of poisonous and side effect, the safety inspection such as blood, stale, shit routine and liver functions found no distinct change, and this embodiments the safety symptom of the natural compound production and it is a complement and extending of the clinical treatment effect of wubabiwuona. It has solved the medicinal puzzle of medicine-proof, addicton when using the insomnia medicine, the puzzle has been longing to be resolved and hasn't been resolved drastically in American physic field.

### BRIEF SUMMARY OF THE INVENTION

After about two hundred years's snub, the use of the plants are getting hotter and hotter. Plants become popular in advanced counties again, and people take ways to keep the health and beauty. On the other hand, because of the heavier environmental pollution, people pay attention to the combination of the drug action and body's autoimmunity when they treat their diseases. The statistic figure suggest that more and more people use the plants to treat diseases in Europe, North America and Australia. In Germany the plant drug market price got to 3,000,000,000 $ in 1993. In England and Spain it increase by 10% and 30%, respectively, so as it in the United States. The americans are not unacquainted with the plant drugs, for example, the popular Coca Cola is made from coca, which is the american plant drug. The original formula of the cocacola was bought in a small store.

The making method about the six kinds of plants and sodium pentobarbital's synergistic action wares is the best and reasonable formula of more than one hundred ones. Under the cautious and strict conditions, extract the plant ingredients, absorb them by WLD resin, separate and purify them, and use the finger mark atlas quantity to make sure that the end product poise and stabilize. The compound wares' pharmacodynamics study prove the pharmacological activity by GJ-7902 triphotictract mouse activity record meter. It can record the mice's activity's times, and test the effect of the compound wares. The mice were injected with pentobabital sodium intraperitoneally. The righting reflexis off for one minute is the index. We want to show that the compound wares and pentobabital sodium have synergistic action under the threshold. The compound wares effect quickly, long, fast and powerful. The high dose makes mostly animals sleep but light, which is inferoir to the same dose pentobabital sodium. The high dose have the effect against the pentylene tetrazol anticonvulsant action. (table 3). We have test in one hundred patients in clinic showed that the compound wares have effects on insomnia, the total effective rate is 95%, the obvious is 83% (table 4).

### BRIEF DESCRIPTION OF THE SEVERAL VIEWS OF THE DRAWING

### Table of Contents

Table 1 The components of the six plant compound and pentobabital sodium's synergistic action

Table 2 The making method and craftwork processing of the six plant compound

Table 3 The pharmacological activity study of the six plant compound and pentobabital sodium's synergistic action

Table 4 The clinical observation of the six plant compound and pentobabital sodium's synergistic action

Table 1 the components of the six plant compound and pentobabital sodium's synergistic action (see attached table 1)

Table 2 The making method and craftwork processing of the six plant compound (see attached table 2)

Table 3 the pharmacological activity study of the six plant compound and pentobabital sodium's synergistic action

The six plant compound wares and pentobabital sodium's synergistic action is used for neurasthenia, asomnia and too much dream, disconcerting, dizzy and anemia. We study its pharmacodynamics and prove the activity.

I. Experiment Materials

1. The six plant compound wares, batch number: 990804

2. Pentobabital sodium (Union)

3. Pentylene tetrazol

II. Method and Result

1. Calm Effect of the Compound

The male mice (weighing 18-21 g) were divided into groups at random. Every group had ten. Experimental group douched the stomach with the compound by 0.1, 0.2 ml/10 g, the control group gave the same volumn saline. Thirty minutes later, the mice were put into GJ-7902 triphotictract mouse activity record meter. After five minutes' adaption, we recorded the mice's activity's times (table 1 was the result). We can concluded that the compound have the calm action.

Table 1 the Calm Effect of the Wares (See Attached Table 3)

2. The synergistic Action of the Compound and the Pentobabital Sodium

The male mice (weighing 18-22 g) were divided into groups at random. Every group had twenty five. Experimental group douched the stomach with the compound by 0.1, 0.2 ml/10 g, the control group gave the same volumn saline. Five minutes later, the mice were injected with pentobabital sodium intraperitoneally (15 mg/kg), then put into the constant temperature infant cabinet at 37° C. We observed the reaction of the mice sleep. The standard was the one minute continous lost righting reflexs (table 2 was the result). We can concluded that compound wares and pentobabital sodium have synergistic action under the threshold at 15 mg/kg. The compound wares effect quickly, long, fast and powerful.

Table 2 The Sleeping Synergistic Action of Compound Wares and Pentobabital Sodium (see Attached Table 4)

3. The Hypnosis Effect of Mian Anning (Taking Orally)

The male white mice (weighing 18-22 g) were divided into groups at random. Every group had twenty five. Experimental group douched the stomach with the compound by 0.08, 0.3, 0.4 ml/10 g, pentobabital sodium 30 mg/kg, the control group gave the same volumn saline. Then put them into the constant temperature infant cabinet at 37° C. We observed the mumber of the mice sleep. The standard was the half minute continous lost righting reflexs (table 3 was the result). We can concluded that high dose (0.4 ml/10 g) compound wares and pentobabital sodium made the animals sleepy but lighter sleep. The pentobabital sodium (30 ml/kg) let them all sleep. The compound wares had hypnosis effect.

Table 3 the Compoud Wars had Effects on Hypnosis (See Attached Table 5)

4. The Compound Wares had the Effects on Convulsion of Pentylene Tetrazol

The white mice (weighing 18-22 g) were divided into groups at random, half male and female. Every group had fifteen. Experimental group douched the stomach with the compound by 0.2, 0.4 ml/10 g, the control group gave the same volumn saline. Forty minutes later, pentylene tetrazol was injected intraperitoneally (125 mg/kg). We regulated the latent period as the time from administration to convulsion, and the continuous peroid as the time from convulsion to death (table 4 was the result). It showed that 0.2 ml/10 g compound wares had no effects on latent and continuous peroid convulsion (p>0.05), and 0.4 ml/10 g compound wares had on effects on the latent peroid, either. But it could extend the continuous peroid (p<0.05). We can concluded that the compound wares had anti-convulsion effects on pentylene tetrazol.

Table 4 the Compound Wares had the Effects on Convulsion of Pentylene Tetrazol (See Attached Table 6)

III. Brief Summary and Discussion

The study of the pharmacodynamics showed the six plant compound wares have good calm effects, and the compoud wares could synergy pentobabital sodium to sleep quickly, long. The wares have the anti-convulsion effects of pentylene tetrazol. It could sedate, hypnotize and synergy pentobabital sodium, and it can be used for neurasthenia, asomnia and too much dream, disconcerting, dizzy and anemia and so on.

Table 4 the Clinical Observation of the Six Plant Compound and Pentobabital Sodium'S Synergistic Action

We have test in one hundred patients in clinic showed that the compound wares have synergistic action to observe the effect and ill effect. The following was the record:

Standard Case:

1. Accord with the diagnosis standard;

2. Age between 18˜65.

Observation Parameter:

I. Check Correlated to the Safety

1. Blood routine

2. Urine routine

3. Excrement routine

4. Liver function

II. Check Correlated to Disease Diagnosis and Effect Verdict

1. Sleeping time and sleeping wakefulness level

2. Psycstate after waking up

III. Observing the Change Between the Pro-Treat and Post-Treat States Observation Method

The Clinic Standard Effect:

1. Fully recover from an illness: the sleeping time recovers normal or sleep for over 6 hours at night, and psycstate is good after waking up.

2. Obvious effect: take a obvious turn for the better, sleeping time increase by over 3 hours, sleeping depth is deeper

3. Effect: the symptom is lighter than before, sleeping time increase by less than 3 hours

4. No effect: Sleeplessness doesn't relieve but aggravate.

Treat Consequence:

1. Clinic Total Effect in Experimental Group

Table 1 Clinic Effect (See Attached Table 7)

Table 2 the Relationship Between the Course and Effect (See Attached Table 8)

From table 2, the effect is more and more obvious to follow the more and more days. After 3 days, the inregra decrease 192, the rate is 9.85%; 5 days 521, 26.7%; 7 days 833, 42.7%; 9 days 1078, 55.3%; 12 days 1312, 67.3%; 14 days 1474, 75.6%. That shows the six plant compound wares can be taken for long. The longer, the better.

Table 3 the Relationship Between the Age and Effect (See Attached Table 9)

From table 3, under 30 years old, the mean mark of the pro-treat is 20.25, and the post-treat is 3.5; from 30 to 39 years old 19.3, 3.7; from 40 to 49 years old 19.1, 4.7; from 50 to 59 years old 19.6, 5.3; above 60 years old 20, 6.1. From the result we can conclude that the older's effect is smaller than the younger, because the older are weaker and weaker. Take them longer, the better.

2. Ill Effects

There is no any ill effect during the observation, the measure of the safety is normal.

IV. Discussion and Brief Summary

1. The six kinds of plants and sodium pentobarbital's synergistic action wares, treated 100 hypnosis, the total and above obvious effect was 96%, 83%, respectively. The wares had good effect on it.

2. There was no any ill effect during the observation, and there was no any change in blood, urine, excrement routine. Took and ate it expediently.

### DETAILED DESCRIPTION OF THE INVENTION

1. The making method about the six kinds of plants and sodium pentobarbital's synergistic action wares is the United State compound patent. The wares must be purified and separated. The compound wares are composed of Radix Salviae Miltiorrhizae, Radix Rehmanniae, Rhizoma Atractylodis Macrocephalae, Radix Polygoni Multiflori, Radix Polygalae, and Fructus Jujubae.

2. The original materials of the six plant compound, every 100 kg with 600 kg, 400 kg, 200 kg water at 100° C. are extracted for 3, 2, 1 hours, respectively. Discard the residue, and put together the filtrate, let them pass by the chromatography column with WLD resin and absorb them, then eluted them by 65% alcohol from the WLD resin, sprayed and dryed them at 85° C., quatity measure with gas chromatography meter. The standard product was done.

3. The studty on the six kinds of plants and sodium pentobarbital's synergistic action proved that they have good calm effects, and the compoud wares could synergy pentobabital sodium to sleep quickly, long. The wares have the anti-convulsion effects of pentylene tetrazol. It could sedate, hypnotize and synergy pentobabital sodium, and it can be used for neurasthenia, asomnia and too much dream, disconcerting, dizzy and anemia and so on. The clinical use proved the vality.

(Attached Table 1)

| | | |
|---|---|---|
| | Total | 100% |
| 1. | Radix Salviae Miltiorrhizae | 27.8% |
| | Labialae | |
| | Salvia Miltiorrhiza Bge. 's root stem | |
| 2. | Radix Rehmanniae | 13.9% |
| | Scrophlariaceae | |
| | Rehmannia Glutinose Libsch. 's root tuber | |
| 3. | Rhizoma Atractylodis Macrocephalae | 11.1% |
| | Compositae | |
| | Atractylides Macrocephala Koidz. 's root stem | |
| 4. | Radix Polygoni Multiflori | 13.9% |
| | Polygonaceae | |
| | Polygonum Multiflorun Thumb. 's vine stem | |
| 5. | Radix Polygalae | 5.5% |
| | Polygalaceae | |
| | Polygala Tenuifolia. 's root | |
| 6. | Fructus Jujubae | 27.8% |
| | Rhamnaceae | |
| | Ziziphus Jujuba Mill. 's fruit | |

(attached table 2)

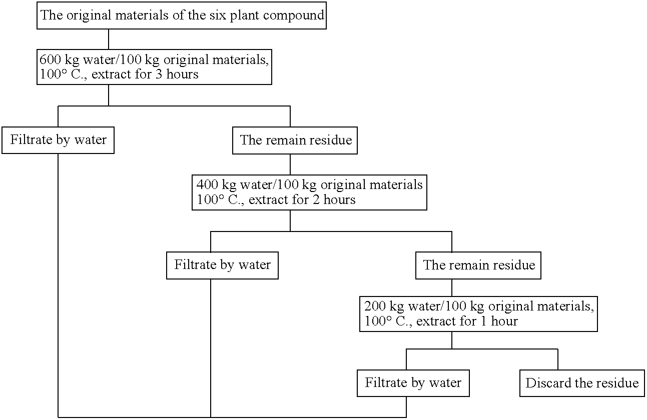

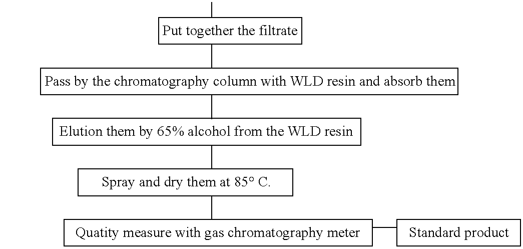

(Attached Table 3)

| Group | Dosage (ml/10 g) | n | Activity times(\overline{X} ± SD) |
|---|---|---|---|
| Saline | The same | 10 | 45.6 ± 26.4 |
| The compound wares | 0.2 | 10 | 8.4 ± 5.6***T |
| The compound wares | 0.1 | 10 | 41.7 ± 18.6* |

*p > 0.05
***p < 0.01 compared with the saline group

(Attached Table 4)

| | Dosage | Pentobabital sodium | | The sleep animals | | | |
|---|---|---|---|---|---|---|---|
| Group | (ml/10 g) | (mg/kg) | n | 5′ | 30′ | 60′ | 90′ |
| Saline | The same | 15 | 25 | 0 | 0 | 0 | 0 |
| Compound wares | 0.2 | | 25 | OTT | 0 | 0 | 0 |
| Compound wares | 0.2 | 15 | 25 | 18*** | 25*** | 8** | 2 |
| Compound wares | 0.2 | 15 | 25 | 10*** | 25* | 5 | 2 |

Note:
**p < 0.05 ***p < 0.01, compared with the saline

(Attached Table 5)

| | Dosage | | The sleep animals | | | |
|---|---|---|---|---|---|---|
| Group | (ml/10 g) | n | 5′ | 30′ | 60′ | 90′ |
| Compound wares | 0.08 | 25 | 0 | 0 | 0 | 0 |
| Compound wares | 0.30 | 25 | 0 | 2 | 0 | 0 |
| Compound wares | 0.40 | 25 | 23*TT | 25*TT | 15*TT | 0 |
| Pentobabital sodium | 30 mg/kg | 25 | 20TT | 25TT | 21TT | 13 |
| saline | The same | 25 | 0 | 0 | 0 | 0 |

Note:
*p > 0.05, compared with pentobabital sodium TTp < 0.01, compared with saline

(Attached Table 6)

| | | | Convulsion time(min)(x ± SD) | |
|---|---|---|---|---|
| | Dosage | | The latent | The continuous |
| Group | (ml/10 g) | n | period | peroid |
| Saline | The same | 15 | 0.7 ± 0.21 | 3.68 ± 2.38 |
| Compound wares | 0.2 | 15 | 0.73 ± 0.28** | 5.32 ± 3.40* |
| Compound wares | 0.4 | 15 | 0.76 ± 0.26* | 6.20 ± 4.18** |

Note:
*p > 0.05
**p < 0.05, compared with saline

(Attached Table 7)

| | Fully | Obvious | No | | Above | |
|---|---|---|---|---|---|---|
| | recover | effect | Effect | effect | obvious effect | Total |
| Case | 39 | 44 | 13 | 4 | 83 | 96 |
| Percent | 39% | 44% | 13% | 4% | 83% | 96% |

From table 1, above obvious effect is 83%, total is 96%, by statistics processing, p<0.01, has significant difference.

(Attached Table 8)

| | Post-treat | | | | | |
|---|---|---|---|---|---|---|
| Pro-treat | 3 days | 5 days | 7 days | 9 days | 12 days | 14 days |

| | | | | | | | |
|---|---|---|---|---|---|---|---|
| Total integra | 1950 | 1758 | 1429 | 1117 | 872 | 638 | 476 |
| Mean integra | 19.5 | 17.58 | 14.29 | 11.17 | 8.72 | 6.38 | 4.76 |
| Decrease rate (%) | | 9.85 | 26.7 | 42.7 | 55.3 | 67.3 | 75.6 |

(Attached Table 9)

| | Total | Mean | Total | | |
|---|---|---|---|---|---|
| | mark | mark | mark | Mean mark | Decrease |
| Age | pro-treat | pro-treat | post-treat | post-treat | rate(%) |
| Under 30 | 81 | 20.25 | 14 | 3.5 | 82.7% |
| 30˜39 | 522 | 19.3 | 100 | 3.7 | 80.8% |
| 40˜49 | 617 | 19.1 | 153 | 4.7 | 75.3% |
| 50˜59 | 392 | 19.6 | 106 | 5.3 | 72.9% |
| Above 60 | 338 | 20 | 103 | 6.1 | 69.7% |

1. I claim “Radix Salviae Miltiorhizae 27.8%, Radix Rehmanniae 13.9%, Rhizoma Atractylodis Macrocephalae 11.1%, Radix polygoni Multiflori 13.9%, Radix Polygalae 5.5%, Fructus Jujupae 27.8%”, the six plant compound and pentobabital's synergistic action and making method are my invention:
That includes the six rude materials, every 100 kg with 600 kg, 400 kg, 200 kg water at 100° C. are extracted for 3, 2, 1 hours, respectively, put together the filtrate, let them pass by the chromatography column with WLD resin, elute them, spray and dry them, quatity measure, the standard product was done.
In my invention, the six plant compound wares have sedate, hypnotize and synergy pentobabital sodium, The clinical use proved the vality.
